(19) United States
(12) Patent Application Publication
(10) Pub. No.: US 2026/0079008 A1
(43) Pub. Date: Mar. 19, 2026

(54) Machine Learning Aided Gait Recognition for Inertial Navigation and Orientation (MARIO)

(71) Applicant: The United States of America, as represented by the Secretary of the Navy, Arlington, VA (US)

(72) Inventors: Minhdao Huynh Nguyen, San Diego, CA (US); Jeffrey Clayton Onners, San Diego, CA (US); Roger Chau Sengphanith, San Diego, CA (US)

(21) Appl. No.: 18/890,249

(22) Filed: Sep. 19, 2024

(51) Int. Cl. G01C 21/16; A61B 5/11; G01C 21/20

(52) U.S. Cl. CPC G01C 21/165; A61B 5/112; G01C 21/20

(57) ABSTRACT

A system capable of determining a pedestrian's position and velocity with respect to a ground surface comprising: a torso IMU mounted to a torso of the pedestrian that is configured to output linear acceleration and angular velocity data (AAV data) associated with the torso; a first leg IMU configured to output linear AAV data associated with the pedestrian's first leg; a second leg IMU configured to output linear AAV data associated with the pedestrian's second leg; a processor mounted to the pedestrian and communicatively coupled to the IMUs, wherein the processor comprises a trained machine learning algorithm configured to estimate the pedestrian's velocity based solely on the linear AAV data from each of the IMUs, or any combination thereof. The processor is configured to use a navigation filter to estimate the pedestrian's position based on the pedestrian's velocity and the linear AAV data from the torso IMU.

### FEDERALLY-SPONSORED RESEARCH AND DEVELOPMENT

The United States Government has ownership rights in the invention claimed herein. Licensing and technical inquiries may be directed to the Office of Research and Technical Applications, Naval Information Warfare Center Pacific, Code 72110, San Diego, CA, 92152; voice (619) 553-5118; NIWC_Pacific_T2@us.navy.mil. Reference Navy Case Number 212258.

### BACKGROUND OF THE INVENTION

Inertial systems may be helpful for pedestrian orientation and navigation in certain environments (e.g., where global navigation satellite system (GNSS) data is not available). There is a need for an improved pedestrian navigation, inertial system.

### SUMMARY

Disclosed herein is a system capable of determining a pedestrian's position and velocity with respect to a ground surface comprising: a torso inertial measurement unit (IMU), first and second leg IMUs, and a processor. The torso IMU is mounted to a torso of the pedestrian and is configured to output linear acceleration and angular velocity data associated with the torso. The first leg IMU is mounted to a first leg of the pedestrian and is configured to output linear acceleration and angular velocity data associated with the first leg. The second leg IMU is mounted to a second leg of the pedestrian and is configured to output linear acceleration and angular velocity data associated with the second leg. The processor is mounted to the pedestrian and communicatively coupled to the torso, first leg, and second leg IMUs. The processor comprises a trained machine learning algorithm configured to estimate the pedestrian's velocity based solely on the linear acceleration and angular velocity data from the torso IMU, the first leg IMU, the second leg IMU, or any combination thereof. The processor is configured to use a navigation filter to estimate the pedestrian's position based on the pedestrian's velocity and the linear acceleration and angular velocity from the torso IMU.

Also disclosed herein is a computer-implemented method for determining a given pedestrian's position and velocity with respect to a ground surface comprising the following steps. A first step provides for collecting a set of ground truth velocity and positioning data based on GNSS data corresponding to a training set of pedestrians traversing different types of terrains wearing GNSS receivers. Another step provides for training a machine learning algorithm with the ground truth velocity and positioning data to generate a trained machine learning algorithm that is stored on a computer. Another step provides for mounting a torso IMU to a torso of the given pedestrian. The torso IMU is configured to output linear acceleration and angular velocity data associated with the torso. Another step provides for mounting a first leg IMU to a first leg of the given pedestrian. The first leg IMU is configured to output linear acceleration and angular velocity data associated with the first leg. Another step provides for mounting a second leg IMU to a second leg of the pedestrian. The second leg IMU is configured to output linear acceleration and angular velocity data associated with the second leg. Another step provides for estimating, with the trained machine learning algorithm, the given pedestrian's velocity based solely on the linear acceleration and angular velocity data from the torso IMU, the first leg IMU, the second leg IMU, or any combination thereof. Another step provides for the processor using a navigation filter to estimate the given pedestrian's position based on the angular velocity from the torso IMU and the given pedestrian's velocity and the linear acceleration as estimated by the trained machine learning algorithm.

### BRIEF DESCRIPTION OF THE DRAWINGS

Throughout the several views, like elements are referenced using like references. The elements in the figures are not drawn to scale and some dimensions are exaggerated for clarity.

FIG. 1 is a side-view drawing of an embodiment of a pedestrian inertial navigation system.

FIG. 2 is a system diagram.

FIG. 3A is a prior art image depicting a single batch for an image classifier embodiment of a CNN.

FIG. 3B is an image depicting a single batch for an embodiment of a machine learning algorithm.

FIG. 4 is a sliding window diagram.

FIG. 5 is an illustration of a pedestrian performing training walks/runs to generate training data for a machine learning algorithm/model.

FIG. 6 is a flowchart of a method.

FIG. 7 is an example process flow of a machine learning algorithm.

FIG. 8 is an example process flow of a machine learning algorithm.

### DETAILED DESCRIPTION OF EMBODIMENTS

The systems and methods disclosed below may be described generally, as well as in terms of specific examples and/or specific embodiments. For instances where references are made to detailed examples and/or embodiments, it should be appreciated that any of the underlying principles described are not to be limited to a single embodiment, but may be expanded for use with any of the other methods and systems described herein as will be understood by one of ordinary skill in the art unless otherwise stated specifically.

References in the present disclosure to “one embodiment,” “an embodiment,” or any variation thereof, means that a particular element, feature, structure, or characteristic described in connection with the embodiments is included in at least one embodiment. The appearances of the phrases “in one embodiment,” “in some embodiments,” and “in other embodiments” in various places in the present disclosure are not necessarily all referring to the same embodiment or the same set of embodiments.

As used herein, the terms “comprises,” “comprising,” “includes,” “including,” “has,” “having,” or any variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or.

Additionally, use of words such as “the,” “a,” or “an” are employed to describe elements and components of the embodiments herein; this is done merely for grammatical reasons and to conform to idiomatic English. This detailed description should be read to include one or at least one, and the singular also includes the plural unless it is clearly indicated otherwise.

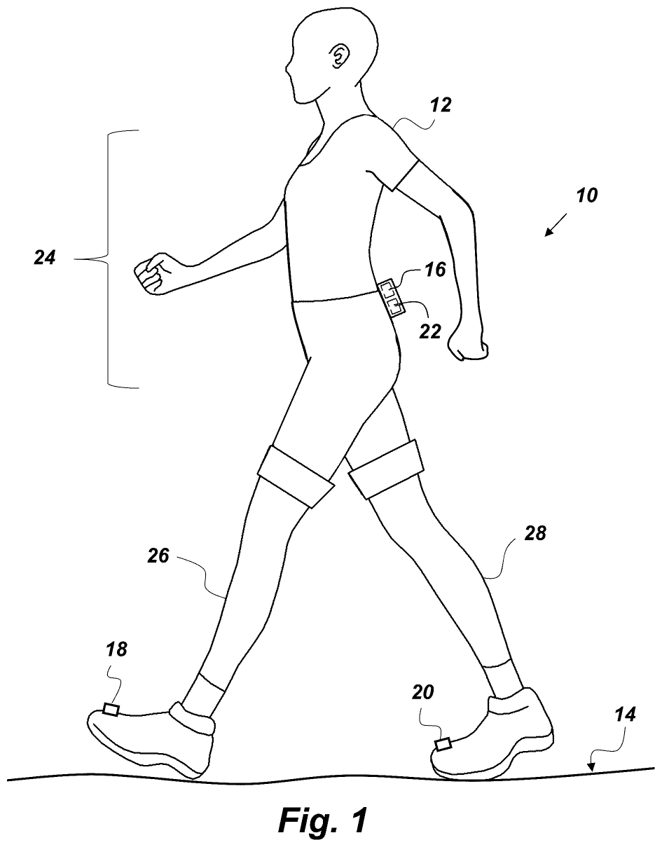

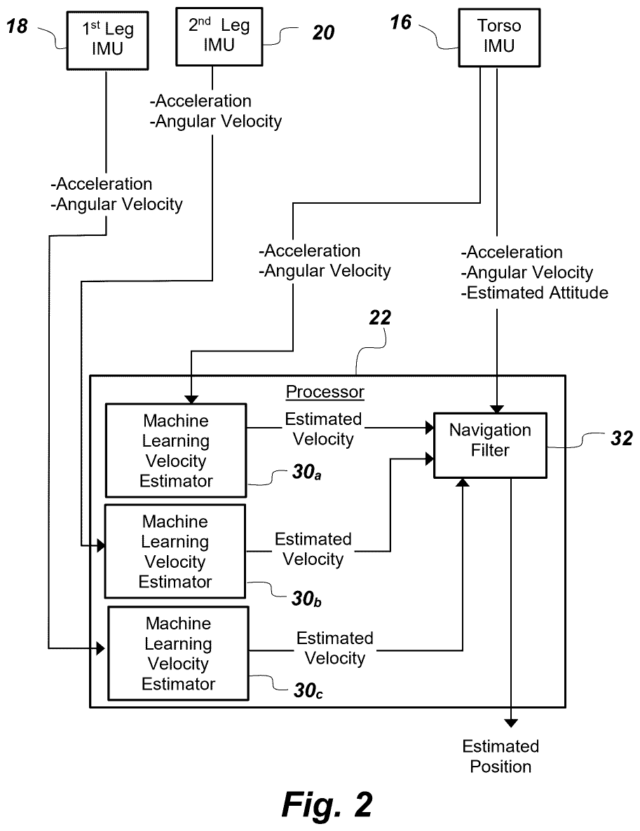

FIG. 1 is a side-view illustration of a system 10 capable of determining a position and velocity of a pedestrian 12 with respect to a ground surface 14 for navigation using data from wearable IMUs. System 10 comprises, consists of, or consists essentially of a torso IMU 16, a first leg IMU 18, a second leg IMU 20, and a processor 22. The torso IMU 16 is mounted to a torso 24 of the pedestrian 12 and is configured to output linear acceleration and angular velocity data associated with the torso 24. The first leg IMU 18 is mounted to a first leg 26 of the pedestrian 12 and is configured to output linear acceleration and angular velocity data associated with the first leg 26. The second leg IMU 20 is mounted to a second leg 28 of the pedestrian 12 and is configured to output linear acceleration and angular velocity data associated with the second leg 28. The processor 22 is mounted to the pedestrian 12 and communicatively coupled to the torso, first leg, and second leg IMUs 18, 20, and 22. It is to be understood that although the processor 22 is shown in FIG. 1 as being positioned on the pedestrian's torso, the processor 22 may be placed on any convenient location on the pedestrian 12. The processor 22 comprises a trained machine learning algorithm 30 (depicted in FIG. 2), one for each IMU worn by the pedestrian. The processor 22 is configured to estimate the pedestrian's velocity based solely on the linear acceleration and angular velocity data from the torso IMU 16, the first leg IMU 18, the second leg IMU 20, or any combination thereof. The processor 22 may be configured to use a navigation filter 32 (depicted in FIG. 2) (e.g., extended Kalman Filter) to estimate the pedestrian's position based on the pedestrian's velocity and the linear acceleration and angular velocity from the torso IMU 16.

FIG. 2 is a system diagram of the system 10. Each of the leg IMUs 18 and 20 are configured to send acceleration and angular velocity data respectively corresponding to the first and second legs 26 and 28 to the machine learning algorithms 30_{c} and 30_{b}. The torso IMU 22 is configured to send angular velocity data corresponding to the pedestrian's torso 24 to the machine learning algorithm 30ₐ. The torso IMU 22 is also configured to send angular velocity data, acceleration data, and torso orientation data to the navigation filter 32. From the data received by the machine learning algorithms 30, the processor 22 is able to estimate an overall pedestrian velocity, which may be fed into the navigation filter, which in turn may calculate a geographical position of the pedestrian 12.

System 10 may be characterized as a velocity-aided inertial navigation system where the velocity is estimated using wearable IMUs mounted on a pedestrian's body. The torso IMU 16 may be mounted anywhere on the pedestrian's torso (e.g., chest, back). For example, in FIG. 1, the torso IMU 22 is shown mounted to a lower back of the pedestrian 12. As mentioned above, the torso IMU 16 may be used to capture the pedestrian's orientation. In FIG. 1, the first and second leg IMUs 18 and 20, which may be used to determine the pedestrian's gait, are shown as being mounted to the pedestrian's feet, but it is to be understood that the first and second leg IMUs 18 and 20 may be mounted on other portions of the pedestrian's legs (e.g., shin, thigh, knee, etc.). As mentioned above, the overall pedestrian velocity estimated by the machine learning algorithm 30 using the acceleration and angular velocity data produced from each IMU, may be fed to the machine learning algorithm 32 to estimate the pedestrian's position.

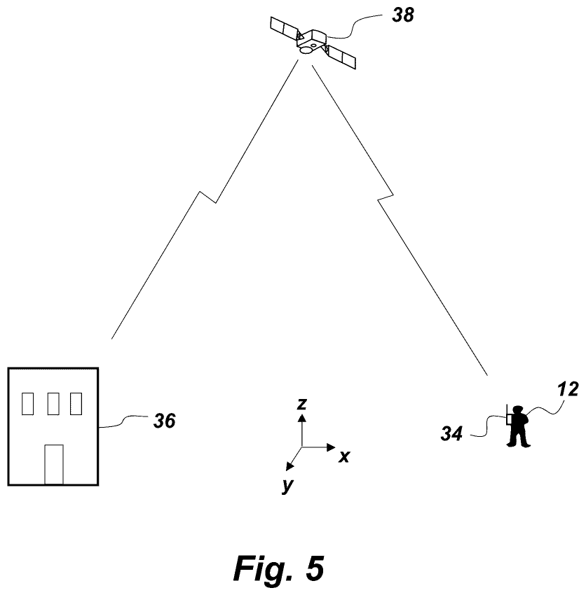

The machine learning algorithm may be a software module stored within processor 22 and may be trained with ground truth data. The ground truth data may be obtained from IMU data and GNSS data from one or more pedestrians performing training walks over different terrains while wearing the torso IMU 16, the first and second leg IMUs 18 and 20, and a GNSS receiver 34 (depicted in FIG. 5). During the training walks, multiple ground truth data runs may be generated by having the pedestrian(s) traverse different terrains with different gaits (e.g., walking, running, shuffling, walking backwards, moving sideways, etc.) while wearing the GNSS receiver 34 and the IMUs. In this way, the machine learning algorithm 30 may be trained to correlate certain IMU data with a certain overall velocity of the pedestrian 12.

Suitable examples of the machine learning algorithm 30 include, but are not limited to, neural networks-including convolutional neural networks (CNNs), long short-term memories (LSTMs), reinforcement learning, and deep learning, decision trees, etc. In one example embodiment of system 10, each machine learning algorithm 30 is a custom Keras-based CNN configured to predict overall pedestrian velocity given IMU data, which was modified to allow layer input shapes to be altered and to allow multi-class outputs as described in further detail below.

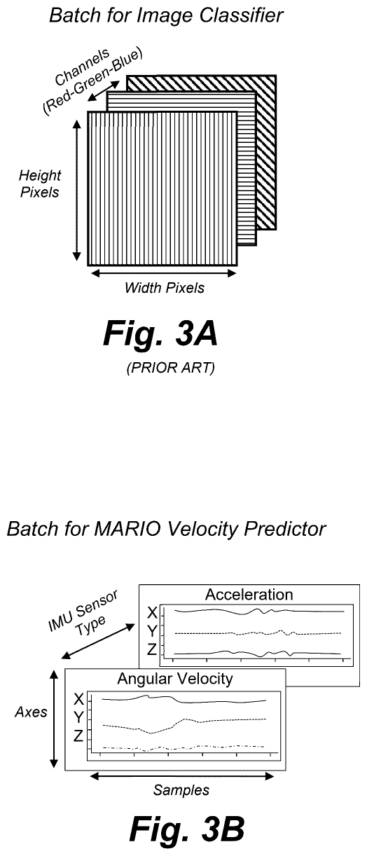

With respect to CNNs, a batch size refers to the number of training examples propagated through the network in a single forward and backward pass. FIG. 3A is a prior art image depicting a single batch for an image classifier embodiment of a CNN. FIG. 3B, in contrast, is an image depicting a single batch for the custom Keras-based CNN embodiment of the machine learning algorithm 30 of system 10, in which embodiment each IMU included two types of IMU sensors (i.e., an accelerometer and a gyroscope) that each produced three axes (X, Y, and Z) of inertial data over a time axis, representing a 2-dimensional (2D) array. Each 2D array (one per sensor) were stacked across a third channel dimension. In one embodiment, small windowed segments of time-series data (0.5 seconds at 100 Hz) constitutes a single batch that were fed into the custom Keras-based CNN, making the final shape (X, 3, 50, 2) where X is the number of batches in the dataset.

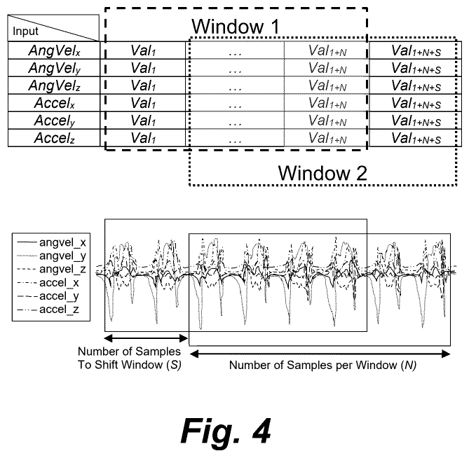

FIG. 4 is a sliding window diagram showing a sliding window approach that may be used to generate pseudo-real-time estimates for both training and testing of the machine learning algorithm 30. The number of samples the windows shift(S) is variable. Overlap between batches occurs when the Sis less than the batch window size as shown in FIG. 4. Including overlap may be desirable during testing to simulate a real-time sliding window. During training, including overlap could cause overfitting due to duplicate data in the training dataset. However, movement patterns will tend to be quick, causing batches with overlap to appear unique, all while having a similar ground truth. This sliding window approach can act similarly to transforming an image to help generalize an image classification network. One embodiment of system 10 uses a train shift value of 25 samples (quarter of a second). For an example of a test file, the first three consecutive batches would take place over intervals t=0 to t=0.49 seconds, t=0.25 to t=0.74 seconds, and t=0.50 to t=0.99 seconds. The batches for training data may be created in the same way and may be shuffled randomly during training to improve generalizability.

FIG. 5 is an illustration of the pedestrian 12 performing training walks/runs in accordance with an example setup that may be used to train the machine learning algorithm 30. A Ublox ZED-F9P GNSS transceiver may be used as the GNSS receiver 34 worn by the pedestrian 12 during training walks/runs in addition to the torso, first leg, and second leg IMUs 16, 18, and 20. The GNSS receiver 34 can output raw GNSS logs up to 10 Hz and also may output a pulse per second (PPS) and time message to synchronize data. In one embodiment, a GNSS post-processed kinematic (PPK) may be used to determine the ground truth data for the system 10. The GNSS PPK may use raw GNSS logs from the GNSS receiver 34 mounted on the pedestrian 12 and raw GNSS logs from a nearby base station 36 to produce a position estimate with an accuracy of +/−two centimeters. This accuracy allows for a good ground truth at pedestrian speeds. The base station 36 preferably should be stationary with a known location. With that data, corrections may be calculated for each GNSS satellite 38 within view of the base station 36. These corrections may be applied to the raw GNSS logs from the user's GNSS receiver 34 with an accuracy of two centimeters. A suitable example of the base station 36 is one of the GNSS base stations of the network of GNSS base stations maintained across North America by the University NAVSTAR Consortium (UNAVCO) that provides the raw GNSS logs and updates the logs daily.

The navigation filter 32 may be any software suite capable of providing a navigation solution from acceleration and angular velocity data from an IMU and an initial absolute position. Suitable examples of the navigation filter 32 include, but are not limited to, an extended Kalman filter, a Rao-Blackwellized Particle filter, and an Unscented Kalman filter. Suitable examples of the first and second leg IMUs 18 and 20 include, but are not limited to, ADIS 16467-3 IMU produced by Analog Devices incorporated in Massachusetts. Each module was mounted on each foot. The ADIS 16467-3 IMU has a range of +/−2000 degrees per second which allows data collection even when the user/pedestrian is running without saturation in the measurements. A suitable example of the torso IMU 16 includes, but is not limited to, a 3DM-CV7-AHRS IMU, manufactured by Microstrain, a Vermont corporation. Table 1 contains training data information collected during several example training walks/runs by three separate pedestrians 12. In this example, the training dataset is composed of a number of different data runs collected over multiple days. For each data run listed in Table 1, each user/pedestrian only moved in the forward-facing direction and did not move sideways, diagonally, or backwards. Also in this example, the user/pedestrian was allowed to make slow or quick turns while moving forward. There were three sites each with different terrain. Site 1 had dirt terrain. Site 2 had grass terrain. Site 3 had road terrain. Data runs were collected with three different users. Table 1 shows the number of runs in the training dataset.

TABLE 1

| Terrain | User A Runs | User B Runs | User C Runs |
|---|---|---|---|
| Dirt | 25 | 5 | 7 |
| Grass | 11 | 5 | 5 |
| Road | 24 | 8 | 7 |

Wide bandwidth IMUs have higher noise than lower bandwidth IMUs. The higher noise can cause higher error in position estimates. Also, a foot mounted IMU has a limited SWAP (size, weight, and power) so larger and more sensitive IMUs should not be used when mounted to a foot. With system 10, the first and second leg IMUs 18 and 20 do not require to be low noise since the torso IMU 16, which may be deemed the main navigation IMU, is mounted to the pedestrian's torso 24 and can be larger than the first and second leg IMUs 18 and 20. The torso-mounted location of the main navigation IMU allows for higher SWAP and better IMUs to be used that will improve pedestrian position estimation.

Additional sensors can be used with system 10 to improve the velocity estimate or the position estimate of the pedestrian 12. For example, a magnetometer can measure earth's magnetic field and can be mounted on the feet and/or lower back of the pedestrian. The magnetometer's output may contribute to velocity, heading, and/or position estimates generated by the processor 22. Also, a temperature sensor can be mounted on the feet and/or lower back and provide additional information to compensate for temperature-dependent IMU measurements. Further, barometric pressure data from a barometer, which can be mounted on the pedestrian's legs and/or torso, may be used by the processor 22 to estimate altitude and to aid in the position estimation and velocity estimation during as the pedestrian 12 changes altitude. In addition to pedestrian-mounted embodiments, system 10 can be applied to other platforms like ships, wheeled vehicles, and airplanes, where the vibrations of the platform can be measured with IMU sensors and machine learning can associate platform velocity with the IMU measurements.

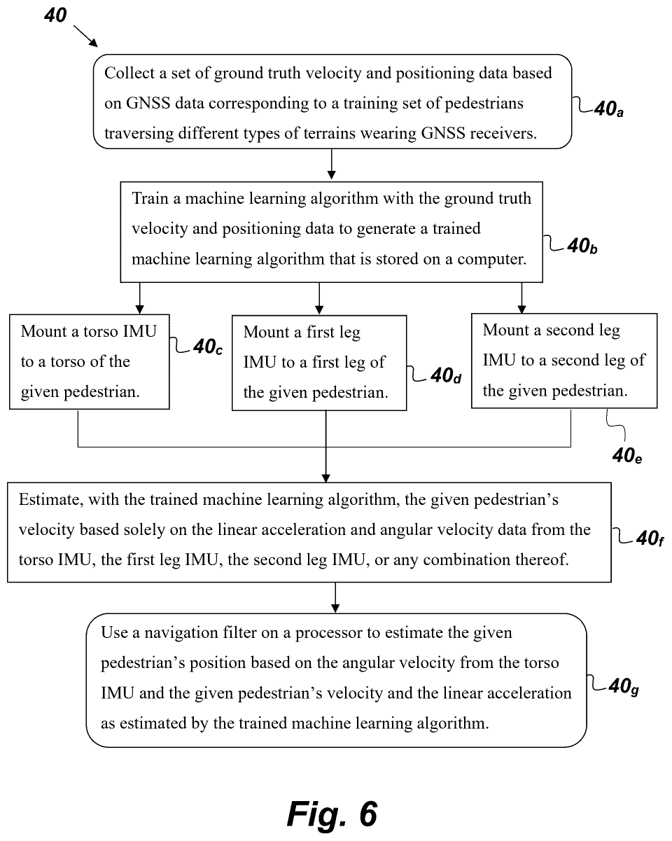

FIG. 6 is a flowchart of a computer-implemented method 40 for determining a given pedestrian's position and velocity with respect to a ground surface comprising the following steps. A first step 40ₐ provides for collecting a set of ground truth velocity and positioning data based on GNSS data corresponding to a training set of pedestrians traversing different types of terrains wearing GNSS receivers. Another step 40_{b} provides for training a machine learning algorithm with the ground truth velocity and positioning data to generate a trained machine learning algorithm that is stored on a computer. Another step 40_{c} provides for mounting a torso IMU to a torso of the given pedestrian. The torso IMU is configured to output linear acceleration and angular velocity data associated with the torso. Another step 40ₐ provides for mounting a first leg IMU to a first leg of the given pedestrian. The first leg IMU is configured to output linear acceleration and angular velocity data associated with the first leg. Another step 40ₑ provides for mounting a second leg IMU to a second leg of the pedestrian. The second leg IMU is configured to output linear acceleration and angular velocity data associated with the second leg. Another step 40_{f} provides for estimating, with the trained machine learning algorithm, the given pedestrian's velocity based solely on the linear acceleration and angular velocity data from the torso IMU, the first leg IMU, the second leg IMU, or any combination thereof. Another step 40_{g} provides for the processor using a navigation filter to estimate the given pedestrian's position based on the angular velocity from the torso IMU and the given pedestrian's velocity and the linear acceleration as estimated by the trained machine learning algorithm.

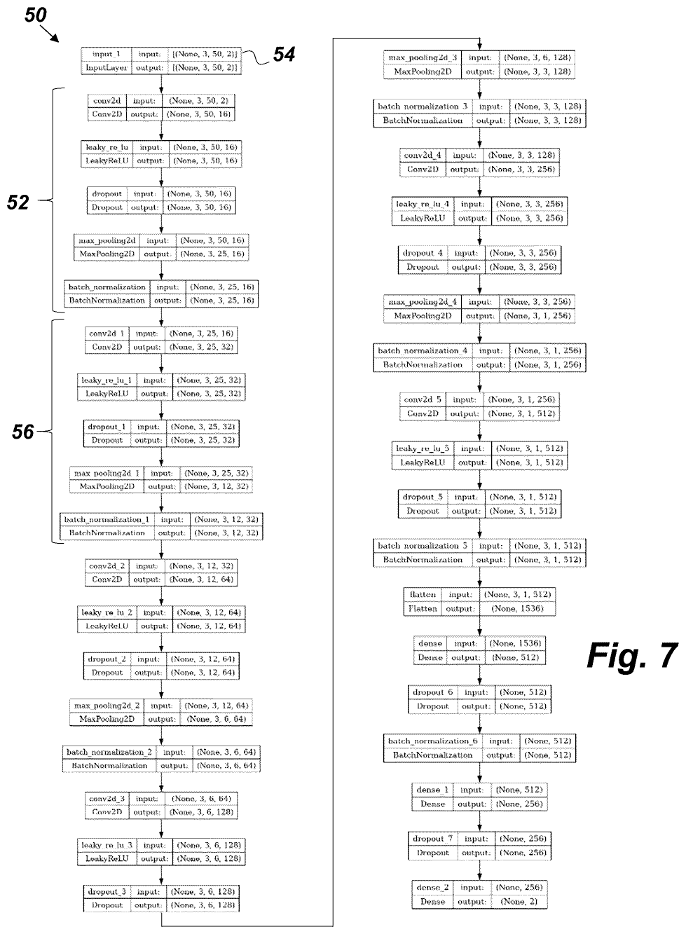

FIG. 7 is an example process flow 50 of an embodiment of the machine learning algorithm 30. This process flow 50 was generated using a keras.utils.plot_model function where a number of convolutional layer blocks (referred to hereafter as Conv-Block Layer) was set to five, and a scalar value (referred to hereafter as a Filter Multiplier value) that determines the number of starting filters used during the first Conv-Block Layer 52 was set to eight. The Filter Multiplier value may be set to any desired scalar value by a user. The number of filters used in each Conv-Block Layer is calculated using that scalar value. The Filter Multiplier may be understood as a factor increase to the channel axis. For example, in the embodiment shown in FIG. 7, after the first Conv-Block Layer 52, each subsequent Conv-Block Layer increases the number of filters by a factor of two. In other words, the Filter Multiplier value does not equal the number of filters used per layer; but rather it is a starting value that affects the total number of filters used per Conv-Block Layer. To illustrate, FIG. 7 shows an input 54 that starts with two channels and with a Filter Multiplier value of eight, the first Conv-Block Layer 52 makes sixteen channels. A second Conv-Block layer 56 doubles the channels to thirty-two and so on. In the example process flow 50 shown in FIG. 7, a custom Keras-based (Tensorflow Library for Python) CNN was used that utilized a combination of the following classes from Keras' Layers API: Input, Conv2D, LeakyReLU, Dropout, MaxPooling2D, BatchNormalization, Flatten, and Dense. The process flow 50 of the machine learning algorithm 30 shown in FIG. 7 utilized the ‘adam’ optimizer and the keras.losses. MeanSquaredError loss function. Also in this embodiment, the ‘LeakyReLU’ activation was used after the ‘Conv2D’ layers, and ‘linear’ activation was used for Dense Layers to allow negative values at the output, representing negative velocities (e.g., moving backwards in an x-direction, or moving left or sideways in a y-direction). A user may be allowed to set number of Conv-Block Layers). Each Conv-Block layer must include at least a Conv2D, LeakyReLU, Dropout, MaxPooling2D, and BatchNormalization layer. The user may also specify a Filter Multiplier value. Changing System 10's Conv-Block Layers and Filter Multiplier allows a user to alter the model size and to compare accuracy and training time. Multiple instances of the machine learning algorithm 30 may be created for each IMU (i.e., one for each IMU worn by the pedestrian 12. For example, the torso IMU 14, the first leg IMU 16, and the second leg IMU 18 may each correspond to a separate trained model, all having an identical ground truth (body velocity). During testing, each IMU/Model will produce a velocity prediction. A weighted average (identical weights used in the process flow 50 shown in FIG. 7) of each prediction is the ‘final’ velocity value, that was sent to a Kalman-filter for position estimation. If one IMU has issues (e.g., battery dies, etc.), its prediction can be ignored in the average, and system 10 will run without issue. Combining the outputs of each IMU into a single machine learning algorithm 30 is also possible (e.g., can be done for comparison).

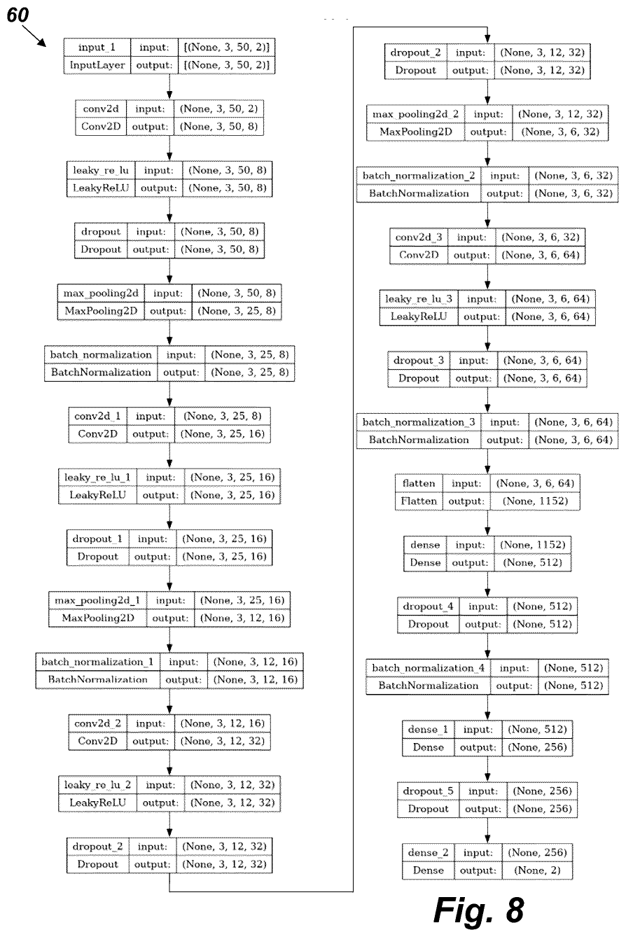

FIG. 8 is an example process flow 60 of an embodiment of the machine learning algorithm 30. Process flow 60 was generated using the keras.utils.plot_model function where the number of convolutional layer blocks was set to three, and the number of filters created per convolutional layer was set to four. Table 2 is a summary of the embodiment of the example machine learning algorithm 30 where the number of convolutional layer blocks was set to five, and the number of filters created per convolutional layer was set to eight. In Table 2 there were 2,497,490 (9.53 MB) total parameters, 2,494,450 (9.52 MB) trainable parameters, and 3,040 (11.88 KB) non-trainable parameters.

TABLE 2

| Layer (type) | Output Shape | Param # |
|---|---|---|
| input_1 (InputLayer) | (None, 3, 50, 2) | 0 |
| conv2d (Conv2D) | (None, 3, 50, 16) | 304 |
| leaky_re_lu (LeakyReLU) | (None, 3, 50, 16) | 0 |
| dropout (Dropout) | (None, 3, 50, 16) | 0 |
| max_pooling2d (MaxPooling2D) | (None, 3, 25, 16) | 0 |
| batch_normalization | (None, 3, 25, 16) | 64 |
| (Batch Normalization) | | |
| conv2d_1 (Conv2D) | (None, 3, 25, 32) | 4640 |
| leaky_re_lu_1 (LeakyReLU) | (None, 3, 25, 32) | 0 |
| dropout_1 (Dropout) | (None, 3, 25, 32) | 0 |
| max_pooling2d_1 (MaxPooling2D) | (None, 3, 12, 32) | 0 |
| batch_normalization_1 | (None, 3, 12, 32) | 128 |
| (BatchNormalization) | | |
| conv2d_2 (Conv2D) | (None, 3, 12, 64) | 18496 |
| leaky_re_lu_2 (LeakyReLU) | (None, 3, 12, 64) | 0 |
| dropout_2 (Dropout) | (None, 3, 12, 64) | 0 |
| max_pooling2d_2 (MaxPooling2D) | (None, 3, 6, 64) | 0 |
| batch_normalization_2 | (None, 3, 6, 64) | 256 |
| (BatchNormalization) | | |
| conv2d_3 (Conv2D) | (None, 3, 6, 128) | 73856 |
| leaky_re_lu_3 (LeakyReLU) | (None, 3, 6, 128) | 0 |
| dropout_3 (Dropout) | (None, 3, 6, 128) | 0 |
| max_pooling2d_3 (MaxPooling2D) | (None, 3, 3, 128) | 0 |
| batch_normalization_3 | (None, 3, 3, 128) | 512 |
| (BatchNormalization) | | |
| conv2d_4 (Conv2D) | (None, 3, 3, 256) | 295168 |
| leaky_re_lu_4 (LeakyReLU) | (None, 3, 3, 256) | 0 |
| dropout_4 (Dropout) | (None, 3, 3, 256) | 0 |
| max_pooling2d_4 (MaxPooling2D) | (None, 3, 1, 256) | 0 |
| batch_normalization_4 | (None, 3, 1, 256) | 1024 |
| (BatchNormalization) | | |
| conv2d_5 (Conv2D) | (None, 3, 1, 512) | 1180160 |
| leaky_re_lu_5 (LeakyReLU) | (None, 3, 1, 512) | 0 |
| dropout_5 (Dropout) | (None, 3, 1, 512) | 0 |
| batch_normalization_5 | (None, 3, 1, 512) | 2048 |
| (BatchNormalization) | | |
| flatten (Flatten) | (None, 1536) | 0 |
| dense (Dense) | (None, 512) | 786944 |
| dropout_6 (Dropout) | (None, 512) | 0 |
| batch_normalization_6 | (None, 512) | 2048 |
| (BatchNormalization) | | |
| dense_1 (Dense) | (None, 256) | 131328 |
| dropout_7 (Dropout) | (None, 256) | 0 |
| dense_2 (Dense) | (None, 2) | 514 |

From the above description of system 10 and method 40, it is manifest that various techniques may be used for implementing the concepts of system 10 and method 40 without departing from the scope of the claims. The described embodiments are to be considered in all respects as illustrative and not restrictive. The method/apparatus disclosed herein may be practiced in the absence of any element that is not specifically claimed and/or disclosed herein. It should also be understood that system 10 and method 40 are not limited to the particular embodiments described herein, but is capable of many embodiments without departing from the scope of the claims.

We claim:

1. A system capable of determining a pedestrian's position and velocity with respect to a ground surface comprising:
a torso inertial measurement unit (IMU) mounted to a torso of the pedestrian, wherein the torso IMU is configured to output linear acceleration and angular velocity data associated with the torso;
a first leg IMU mounted to a first leg of the pedestrian, wherein the first leg IMU is configured to output linear acceleration and angular velocity data associated with the first leg;
a second leg IMU mounted to a second leg of the pedestrian, wherein the second leg IMU is configured to output linear acceleration and angular velocity data associated with the second leg;
a processor mounted to the pedestrian and communicatively coupled to the torso, first leg, and second leg IMUs, wherein the processor comprises a trained machine learning algorithm configured to estimate the pedestrian's velocity based solely on the linear acceleration and angular velocity data from the torso IMU, the first leg IMU, the second leg IMU, or any combination thereof, and
wherein the processor is configured to use a navigation filter to estimate the pedestrian's position based on the pedestrian's velocity and the linear acceleration and angular velocity from the torso IMU.

2. The system of claim 1, wherein the torso IMU is mounted to a lower back of the pedestrian.

3. The system of claim 2, wherein the first and second leg IMUs are respectively mounted to a first foot and a second foot of the pedestrian.

4. The system of claim 3, wherein the navigation filter is an extended Kalman filter.

5. The system of claim 3, wherein the navigation filter is a Rao-Blackwellized Particle filter.

6. The system of claim 3, wherein the navigation filter is an unscented Kalman filter.

7. The system of claim 4, wherein the machine learning algorithm is a convolutional neural network.

8. The system of claim 7, wherein the trained machine learning algorithm is trained with a dataset that includes ground truth data from a GNSS receiver worn by a training pedestrian during training walks/runs, wherein the training pedestrian also wears the torso, first leg, and second leg IMUs during the training walks/runs.

9. The system of claim 8, wherein the ground truth data is determined by a processor using a GNSS post-processed kinematic (PPK).

10. The system of claim 9, wherein the GNSS PPK is configured to use raw GNSS logs from the GNSS receiver and raw GNSS logs from a base station.

11. A computer-implemented method for determining a given pedestrian's position and velocity with respect to a ground surface comprising:
collecting a set of ground truth velocity and positioning data based on global navigation satellite system (GNSS) data corresponding to a training set of pedestrians traversing different types of terrains wearing GNSS receivers;
training a machine learning algorithm with the ground truth velocity and positioning data to generate a trained machine learning algorithm that is stored on a computer;
mounting a torso inertial measurement unit (IMU) to a torso of the given pedestrian, wherein the torso IMU is configured to output linear acceleration and angular velocity data associated with the torso;
mounting a first leg IMU to a first leg of the given pedestrian, wherein the first leg IMU is configured to output linear acceleration and angular velocity data associated with the first leg;
mounting a second leg IMU to a second leg of the pedestrian, wherein the second leg IMU is configured to output linear acceleration and angular velocity data associated with the second leg;
estimating, with the trained machine learning algorithm, the given pedestrian's velocity based solely on the linear acceleration and angular velocity data from the torso IMU, the first leg IMU, the second leg IMU, or any combination thereof; and
using, with the computer, a navigation filter to estimate the given pedestrian's position based on the angular velocity from the torso IMU and the given pedestrian's velocity and the linear acceleration as estimated by the trained machine learning algorithm.

12. The method of claim 11, wherein the computer is configured to use a sliding window approach to generate pseudo-real-time estimates for both training and testing the machine learning algorithm.

13. The method of claim 12, wherein the step of estimating further comprises:
representing the linear acceleration and angular velocity data from each of the first and second leg IMUs and the torso IMU as a separate two-dimensional (2D) array;
stacking the 2D arrays across a third channel dimension; and
feeding small windowed segments of time-series data generated during the sliding window approach into the trained machine learning algorithm.

14. The method of claim 12, wherein, during testing, the sliding window approach comprises shifting the window by a number of samples S that overlaps with a previous window or windows.

15. The method of claim 14, wherein the training step is performed with a dataset that includes ground truth data from a GNSS receiver worn by a training pedestrian during training walks/runs, wherein the training pedestrian also wears the torso, first leg, and second leg IMUs during the training walks/runs.

16. A computer-implemented method for training a machine learning model to determine a given pedestrian's position and velocity with respect to a ground surface comprising:
collecting a set of ground truth velocity and positioning data from GNSS receivers worn by pedestrians traversing different types of terrains;
collecting IMU data from IMUs mounted to each pedestrian's legs and torso, wherein the IMU data is time-correlated to the ground truth velocity and positioning data;
training a separate machine learning model for each of the IMUs mounted to the pedestrian by:
using a stochastic gradient descent method based on adaptive estimation of first-order and second-order moments, such as an Adam optimizer, to perform convolutions using windows over segments of the IMU data to discover common features within the IMU data;
wherein each window over a segment of time-series IMU data corresponding to a given IMU constitutes a single batch that is fed into the machine learning model corresponding to the given IMU, and wherein each batch consists of a 2D array consisting of three axes of IMU data from the given IMU over a time axis stacked across a third channel dimension;
wherein, each machine learning model is configured to produce a velocity prediction;
generating a final velocity value by determining, with the computer, a weighted average of the velocity predictions from machine learning models; and
using, with the computer, a navigation filter to estimate the given pedestrian's position and velocity with respect to the ground surface based only on the final velocity value and a known starting position.

17. The method of claim 16, further comprising:
allowing a user to set a number of convolutional layer blocks (Conv-Block Layers), wherein each Conv-Block Layer comprises a Conv2D, LeakyRelu activation, Dropout, MaxPooling2D, and BatchNormalization layer; and
allowing a user to alter Filter Multiplier value that determines a number of filters used per Conv-Block Layer.

18. The method of claim 17, wherein each Conv-Block Layer uses a LeakyReLU activation function after a Conv2D layer.

19. The method of claim 18, further comprising using linear activation for Dense Layers to allow negative values for each velocity prediction, representing negative velocities (such as the given pedestrian moving backwards).

20. The method of claim 19, wherein the final velocity value is a weighted average of the velocity predictions from all the machine learning models, and each velocity prediction is weighted equally.
